(19) United States
(12) Patent Application Publication
(10) Pub. No.: US 2021/0183263 A1
(43) Pub. Date: Jun. 17, 2021

(54) GAIT FACILITATION AND FEEDBACK METHOD AND SYSTEM

(71) Applicant: Jacob Virgo, Munster, IN (US)

(72) Inventor: Jacob Virgo, Munster, IN (US)

(21) Appl. No.: 16/711,137

(22) Filed: Dec. 11, 2019

(51) Int. Cl. G09B 19/00; F21V 33/00; F21V 23/02; G09B 5/06

(52) U.S. Cl. CPC G09B 19/003; F21V 33/0064; A61B 5/112; G09B 5/06; F21V 23/02

(57) ABSTRACT

Gait facilitation and feedback methods and systems capable of providing automatic external cues to facilitate improved gait patterns and increased safety with ambulation. During ambulation of a user over a surface, the system generates and simultaneously projects multiple light beams on the surface in front of the user's line of progression. The light beams project multiple illuminated lines in front of and perpendicular to the user's line of progression during ambulation to thereby project a field projection on the surface that is continuously seen by the user during ambulation and is not dependent on stepping of the user during ambulation.

### CROSS-REFERENCE TO RELATED APPLICATIONS

This application claims the benefit of U.S. Provisional Application No. 62/778,047 filed Dec. 11, 2018, the contents of which are incorporated herein by reference.

### BACKGROUND OF THE INVENTION

The present invention generally relates to a gait facilitation and feedback system capable of providing automatic external cues to facilitate improved gait patterns and increased safety with ambulation.

Currently, commercial systems exist that are intended to improve ambulation in people with parkinsonian symptoms. These systems include walkers and canes with a laser line perpendicular to the user's progression, metronomes to provide auditory cues for stepping tempo, and walking programs that are either independent without cues or feedback or are manually guided walking sessions. Physical therapy also plays a large role in working with individuals with parkinsonian symptoms and often uses different cuing and feedback mechanisms in the clinic to improve gait pattern and ambulation. Some strategies used are: auditory clapping, tape lines on the floor, large exaggerated movements with verbal cues, stepping toward or over objects, or treadmill training. These techniques are beneficial in the clinic, but unfortunately do not carry over well into the individual's ambulation once the external stimulus is removed. Currently, it is common for patients to put tape lines on the floor of their home as external cues to assist with ambulation therapy, but tape may not be effective in times of low lighting such as in the middle of the night.

### BRIEF DESCRIPTION OF THE INVENTION

The present invention provides gait facilitation and feedback methods and systems capable of providing automatic external cues to facilitate improved gait patterns and increased safety with ambulation.

According to one aspect of the invention, a gait facilitation and feedback method is provided that includes, during ambulation of a user over a surface, generating and simultaneously projecting multiple light beams on the surface in front of the user's line of progression during ambulation, the light beams projecting multiple illuminated lines in front of and perpendicular to the user's line of progression during ambulation to thereby project a field projection on the surface that is continuously seen by the user during ambulation and is not dependent on stepping of the user during ambulation.

Another aspect of the invention is a gait facilitation and feedback system equipped with means for generating and simultaneously projecting multiple light beams on a surface in front of a user's line of progression during ambulation by the user over the surface so that multiple illuminated lines are illuminated that are parallel to each other in front of and perpendicular to the user's line of progression during ambulation to thereby project a field projection on the surface that is continuously seen by the user during ambulation and is not dependent on stepping of the user during ambulation.

Technical aspects of the methods and systems described above preferably include the ability to continuously provide gait facilitation and feedback by continuously providing visual cuing for proper gait sequencing, and in preferred embodiments also provide real time feedback to track gait pattern performance. The methods and systems may utilize different options for visual and auditory feedback, with the general intent of providing visual cues along a user's walking path, optionally while recording and providing auditory cues and/or feedback on gait pattern performance based on heel and forefoot weight bearing. The methods and systems can be used as a training and management tool for individuals that benefit form external cues, such as individuals that suffer from parkinsonian symptoms, but can be used by essentially any individual undergoing gait training or rehabilitation.

Other aspects and advantages of this invention will be appreciated from the following detailed description.

### BRIEF DESCRIPTION OF THE DRAWINGS

FIGS. 1A and 1B schematically represent front and rear views showing a gait facilitation and feedback system in use by a user during ambulation in accordance with a nonlimiting embodiment of this invention, wherein the system continuously provides visual cues to the user by generating and projecting light beams on a surface in front of the user's line of progression during ambulation, and the light beams project multiple illuminated lines that are in front of and perpendicular to the user's line of progression to thereby project a field projection on the surface that can be continuously seen by the user during ambulation to facilitate stepping and to reduce the tendency for freezing of gait.

FIG. 2 schematically represents the gait facilitation and feedback system of FIG. 1 further equipped with means for sensing weight bearing at the heel and forefoot of a shoe of the user to detect weight bearing at areas of the user's foot at heel contact and mid to late stance phase of a gait cycle during ambulation of the user, and means for generating and transmitting an auditory cue to the user to notify the user that the user has accomplished heel strike and forefoot weight transfer.

FIGS. 3A and 3B schematically represent, respectively, front and rear views of a laser and hologram unit adapted for generating and projecting the light beams of any of the systems of FIGS. 1 and 2.

FIG. 4 schematically represents gait facilitation and feedback systems deployed in rooms in accordance with another nonlimiting embodiment of this invention.

FIG. 5 schematically represents the laser/hologram unit of FIGS. 4A and 4B as a component of a user's belt consistent with the gait facilitation and feedback system of FIGS. 1 and 2.

FIG. 6 schematically represents locations of heel and forefoot sensor pads adapted to be placed in a user's shoe in accordance with an optional aspect of this invention.

FIGS. 7A and 7B schematically represent the laser/hologram unit of FIGS. 4A and 4B as a component of a user's shoe in accordance with an optional aspect of this invention.

### DETAILED DESCRIPTION OF THE INVENTION

The invention will be described below as encompassing gait facilitation and feedback methods and systems capable of providing visual cues (optionally in combination with other automatic external cues) that can be continuously seen by a user of the system to facilitate stepping, reduce the tendency for freezing of gait, facilitate improved gait patterns, and increase safety of the user during ambulation. In certain embodiments, the system may also provide feedback on real-time performance, as well as objective information that can be used for goal setting and provide a reward system to the user. The system may have a high benefit for individuals with Parkinson's or parkinsonian symptoms, as it provides external cues adapted to make up for the loss of internal cuing for motor planning that is seen in these individuals. The reward system is particularly beneficial for this population due to the lack of dopamine in the brain providing reward satisfaction and motivation.

The system is also useful for balance and gait training within different therapeutic regimens. The system may be beneficial for anyone who is working on their gait pattern, ambulation, and balance, as it provides cuing for stepping and feedback on appropriate weight shift. Such use is not limited to the adult or neurologically involved populations, but may be beneficial to pediatric populations as well. The system may be used to help facilitate proper gait patterns in individuals who are working toward decreasing “toe walking,” as this system has the capability of providing a programable visual stimulus in front of the user, can control auditory stimuli (songs, sounds, etc.), and provide weight bearing feedback in real time. The system can be offered with different components and variations of complexities and feedback systems that allow for more affordability across a given demographic and varied use in different environments and situations. The system can be individually used and owned by the user, as well as can be owned and used by an organization, such as a therapy clinic, for in-clinic training, as well as renting to patients for long-term home training.

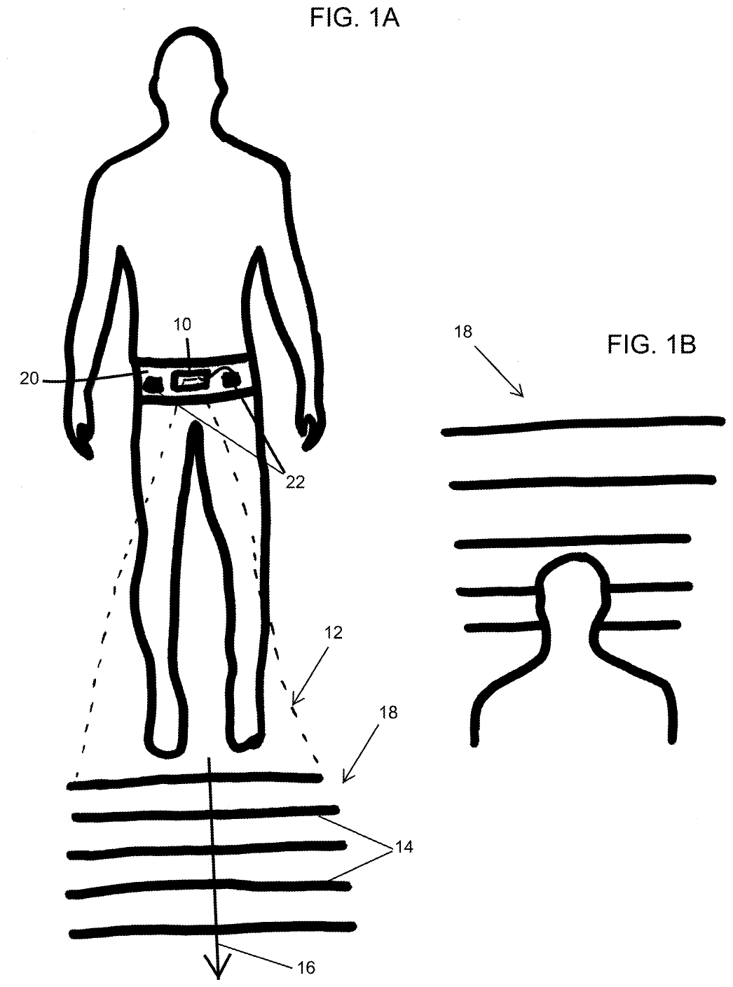

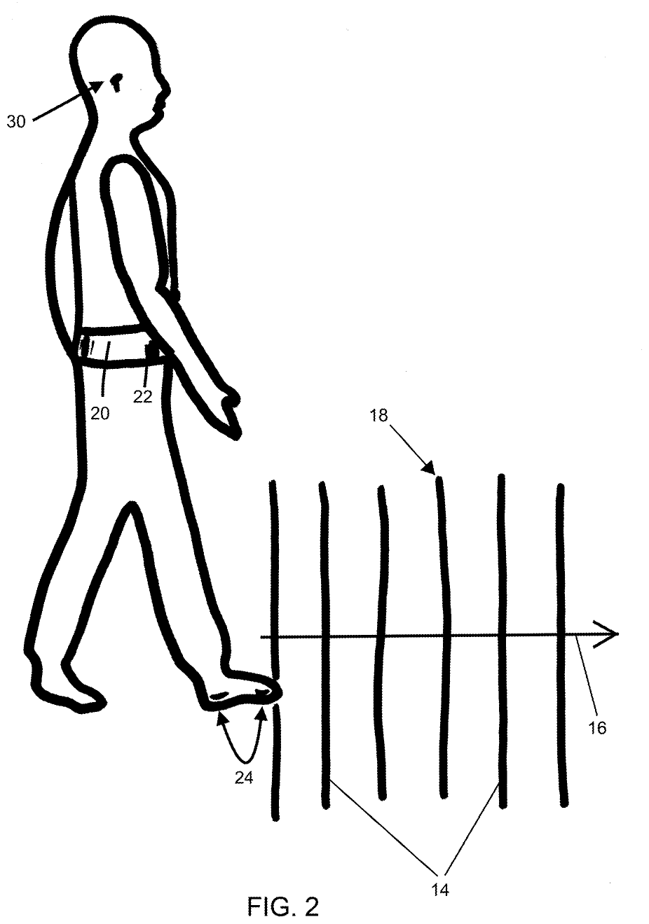

Nonlimiting embodiments of gait facilitation and feedback systems and the use thereof by an individual are represented in FIGS. 1 and 2. In each case, the system comprises a wearable light-generating unit 10 as a means for generating and simultaneously projecting multiple light beams 12 during ambulation of a user/wearer over a surface. Such a unit 10 may be, for example, a laser and hologram device that simultaneously generates multiple laser light beams. In the embodiments of FIGS. 1 and 2, the light beams 12 project multiple illuminated lines 14 on a surface that are parallel to each other and in front of and perpendicular to the user's line of progression 16 during ambulation to thereby project a field projection 18 on the surface that can be continuously seen by the user during ambulation. The operation of the light-generating unit 10 and the generation of the illuminated lines 14 are not dependent on stepping of the user during ambulation, but instead continuous during operation of the unit 10. As a result of comprising multiple illuminated lines 14, the field projection 18 provides a visual cue that the user steps toward or over to improve step length, foot clearance, and heel strike, so that the field projection 18 is able to facilitate stepping by the user and reduce the user's tendency for freezing of gait. Though a laser/hologram unit that generates laser light beams is believed to be preferred as the light-generating unit 10, the use of other light generating devices and other forms of light are foreseeable as long as the illuminated lines 14 are sufficiently visible and distinct to the particular user.

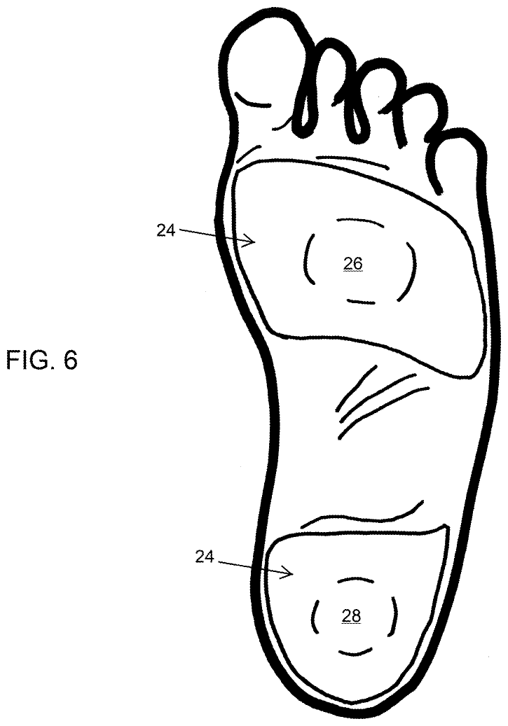

In FIG. 1, the light-generating unit 10 is attached to a belt 20 at or near the center of the belt 20. A battery pack 22 is attached to the belt 20 and is connected to the light-generating unit 10 via a wire connection. In FIG. 2, the system is further equipped with means for sensing weight bearing at the heel and forefoot of a shoe of a user to detect weight bearing at areas of the user's foot at heel contact and mid to late stance phase of a gait cycle during ambulation of the user, and a sound-generating unit for generating and transmitting an auditory cue to the user to notify the user that the user has accomplished heel strike and forefoot weight transfer as sensed by the sensing means. The sensing means are represented in FIG. 2 as placed in the users shoes at the heel and forefoot, and are represented in FIG. 6 as sensor pads 24 that conform to the shape of the heel and forefoot insole and may be attached to the insole of the user's shoes with adhesive. Sensing elements 26 and 28 carried by the sensor pads 24 can be shaped and designed to detect weight bearing at the appropriate areas of the foot at heel contact and mid to late stance phase of the gait cycle. The sensing elements 26 and 28 detect a percent weight bearing at the heel strike and late midstance portions of the gait cycle. When enough weight is detected, the outputs of the sensor elements 26 and 28 may be used by the sound-generating unit to generate and transmit an auditory cue (sound) to the user to identify a successful heel strike and successful midfoot weight transfer during the stance portion of the gait cycle. In the example of FIG. 2, the outputs of the sensor elements 26 and 28 are wirelessly transmitted to earbuds 30 to generate an auditory sound, though the use of other forms of speakers is foreseeable. With this approach, the outputs of the sensor elements 26 and 28 may be directly wirelessly transmitted to the earbuds 30, or the belt 20 may carry a sound generating unit that converts the outputs of the sensor elements 26 and 28 to an auditory signal that is then relayed to the earbuds 30. The auditory cue may be programed into the system through an online application to allow the user to customize the sounds that are generated. This may be a click or something such as music or sounds that could be used as motivation in pediatric users. Alternatively or in addition, the heel and forefoot sensor pads 24 may be adapted to directly provide a mechanical auditory function, for example, a mechanic audible feedback (e.g., “pop” or “click”) when sufficient weight bearing is sensed at the heel and midfoot.

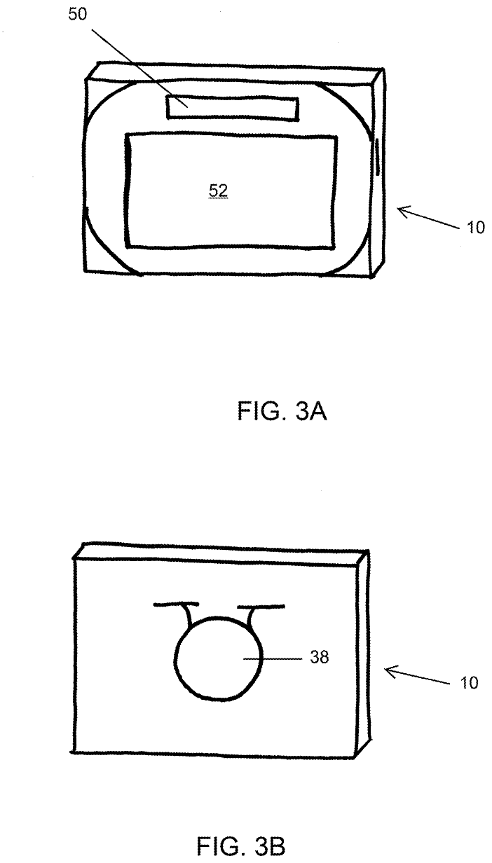

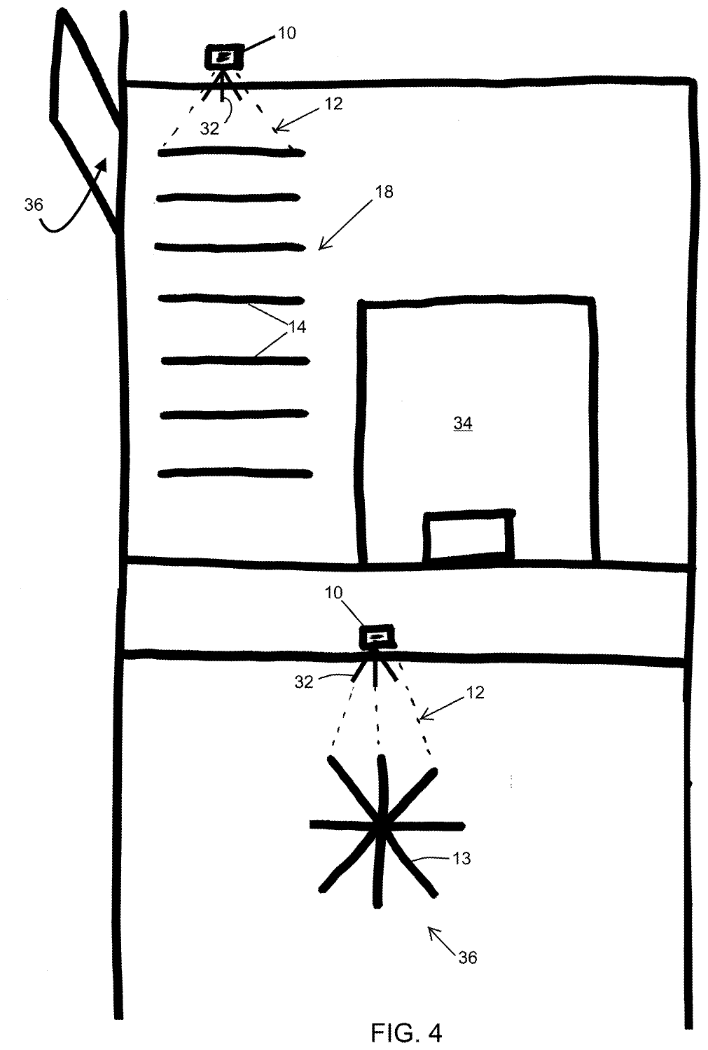

FIGS. 3A and 3B schematically represent front and rear views of a nonlimiting example of the light-generating unit 10. FIG. 3A schematically depicts areas 50 and 52 of the unit 10 from which, respectively, the laser and hologram fields are emitted from the unit 10. The unit 10 is able to be worn on the belt 20 of the user (FIGS. 1 and 2) or mounted on a floor in a desired location (FIG. 4). The unit 10 may be attached to the belt 20 via a click-in ball and socket joint, of which FIG. 3B shows the ball (male end) 38 of such a joint on the back side of the unit 10. With custom design options of the unit 10, a user or clinician can design different patterns of field projections that can be used for exercises, in addition to ambulation therapy. The field projection 18 generated with the unit 10 is preferably controllable independent of the optional sensor pads 24, and though intended for use with the sensor pads 24 could be used separately.

It is also foreseeable that the light-generating unit 10 of FIGS. 3A and 3B could be mounted within any given environment (e.g., the user's hallway from bed to bathroom, doorways to prevent mobility freezing, or even in therapy clinics) to provide functions such as targets, agility ladders, or other field patterns that are commonly created by applying tape to a floor. FIG. 4 is a schematic plan view showing light-generating units 10 in a freestanding form for use in a therapeutic environment or the user's home. In FIG. 4, two units 10 are individually mounted on tripods 32 that can be set up to project the multiple light beams 12 to generate multiple illuminated lines 14 on the floor of a desired location. The resulting projection field 18 may be programable through an online application to allow for various options, such as perpendicular lines from the user's bed 34 to the bathroom 36, or a star pattern 36 projected on the floor of a therapy clinic.

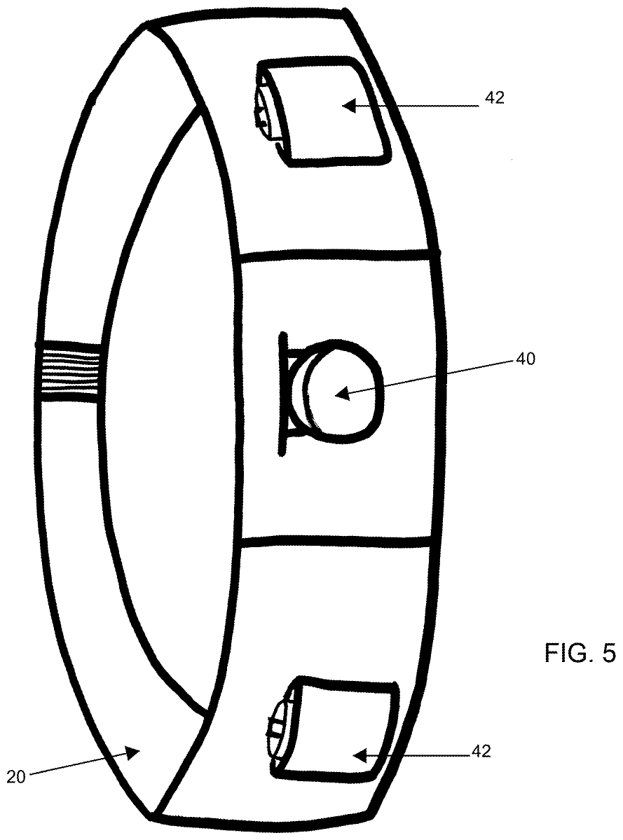

FIG. 5 schematically represents a more detailed view of the belt 20 of FIGS. 1 and 2. The belt 20 is preferably formed of an elastic material and adapted to be worn and attached around the user's waist. The belt 20 is shown as having an anchoring site comprising a socket (female end) 40 of a ball and socket joint for coupling with the complementary ball 38 of the unit 10 in FIG. 3B. The anchoring site may include a built-in gyroscope within the socket 40 to prevent the user's movements from moving the light-generating unit 10 and its field projection 18. The belt 20 is also schematically represented as storing batteries 42 that can be interchanged with those of the light-generating unit 10. Alternatively, the belt 20 and light-generating unit 10 can be equipped with wiring that allows the light-generating unit 10 to be selectively connected to the batteries 42 carried on the belt 20, as shown in FIG. 2. The belt 20 may optionally also mount other components of the system, for example, a previously-noted sound-generating unit that may relay or produce an auditory sound to identify a successful heel strike and midfoot weight transfer.

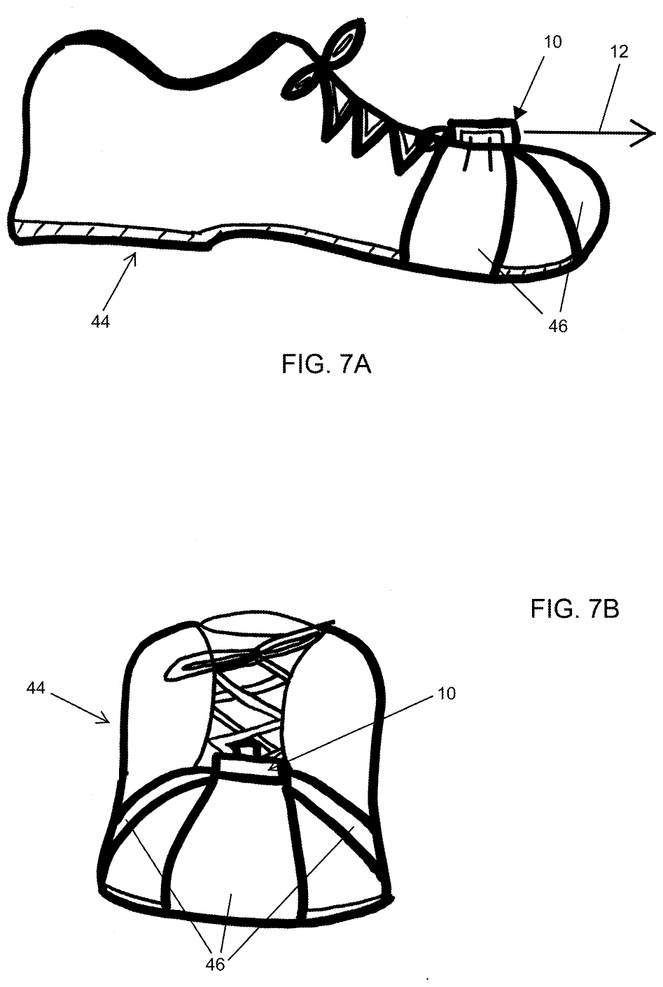

FIGS. 7A and 7B schematically represent the light-generating unit 10 as a component of a shoe attachment 46 adapted to be attached to the top of a user's shoes 44 near the toe. In this embodiment, the laser beam 12 (or another form of narrow light beam) is generated at the level of the toe perpendicular to the user's line of progression 16. Shoe attachments of the type represented in FIGS. 7A and 7B are preferably attached to both of the user's shoes.

As noted previously, the system may operate in combination with an online application that can be accessed on the user's phone or other mobile electronic device. The application can be configured to provide feedback on total steps versus heel strikes and adequate forefoot weight transfer, and allow the user to set goals such as daily heel strike goals that could potentially provide a reward system for the user.

In view of the above, the gait facilitation and feedback system is adapted to detect heel strike and forefoot weight transfer during the gait cycle of a user and have an option to provide auditory feedback to the user, for example, via a mechanical “click” or “pop” sound generated with the sensor pads, or an electric signal generated by the sensor pads (or another sound generating unit) and transmitted to earbuds worn by the user when those tasks are accomplished. Within an online application and/or wireless earbuds or speaker, it is also an option to program music or a metronome to provide auditory cuing rather than feedback or to trigger such cues based on weight bearing inputs from the sensor pads. The system also provides visual cues by means of field projections that contain targets perpendicular to the user's progression for stepping cues and motor freezing prevention. The user's performance can be tracked on an electronic application providing objective information of number of heel strikes and forefoot weight transfers versus total steps that can also be used for goal setting, providing a reward system for the user.

In contrast to conventional commercial ambulation therapy systems, gait facilitation and feedback systems of the present invention are preferably capable of providing comprehensive external cuing and gait feedback wherever the user may be, e.g., whether at a clinic or at home. Such a system can provide visuals cues, auditory feedback/cues, and objective measures/rewards, that can be used personally or as a training tool by therapists. The light-generating unit 10 can also provide several functions, whether it is used at the user's belt buckle for ambulation or exercises, or to provide designs in the clinic or an individual's household.

Gait facilitation and feedback systems of the present invention provide means for using current strategies independently and long-term. With the given evidence of the benefits of prolonged ambulation, use of external cues and providing reward systems for the given population, potential for this system to be highly beneficial is great.

While the invention has been described in terms of a particular embodiment, it should be apparent that alternatives could be adopted by one skilled in the art. For example, the system and its components could differ in appearance and construction from the embodiment described herein and shown in the drawings, and functions of certain components of the system could be performed by components of different construction but capable of a similar (though not necessarily equivalent) function. As such, it should be understood that the above detailed description is intended to describe the particular embodiment represented in the drawings and certain but not necessarily all features and aspects thereof, and to identify certain but not necessarily all alternatives to the represented embodiment and described features and aspects. As a nonlimiting example, the invention encompasses additional or alternative embodiments in which one or more features or aspects of the disclosed embodiment could be eliminated. Accordingly, it should be understood that the invention is not necessarily limited to any embodiment described herein or illustrated in the drawings, and the phraseology and terminology employed above are for the purpose of describing the illustrated embodiment and do not necessarily serve as limitations to the scope of the invention. Therefore, the scope of the invention is to be limited only by the following claims.

1. A gait facilitation and feedback method comprising:
during ambulation of a user over a surface, generating and simultaneously projecting multiple light beams on the surface in front of the user's line of progression during ambulation, the light beams projecting multiple illuminated lines in front of and perpendicular to the user's line of progression during ambulation to thereby project a field projection on the surface that is continuously seen by the user during ambulation and is not dependent on stepping of the user during ambulation.

2. The gait facilitation and feedback method of claim 1, wherein the light beams are generated and projected with a unit supported on the surface.

3. The gait facilitation and feedback method of claim 1, wherein the light beams are generated and projected with a unit mounted on a belt worn by the user.

4. The gait facilitation and feedback method of claim 1, wherein the light beams are generated and projected with a unit attached to a shoe of the user.

5. The gait facilitation and feedback method of claim 1, further comprising sensing weight bearing at the heel and forefoot of a shoe of a user to detect weight bearing at areas of the user's foot at heel contact and mid to late stance phase of a gait cycle during ambulation of the user;

6. The gait facilitation and feedback method of claim 1, further comprising generating and transmitting an auditory cue to the user to notify the user that the user has accomplished heel strike and forefoot weight transfer.

7. The gait facilitation and feedback method of claim 1, wherein the auditory cue is transmitted to the user via a Bluetooth wireless earbud or speaker.

8. A gait facilitation and feedback method comprising:
during ambulation of a user over a surface, generating and simultaneously projecting multiple light beams on the surface in front of the user's line of progression during ambulation, the light beams projecting multiple illuminated lines that are parallel to each other and in front of and perpendicular to the user's line of progression during ambulation to thereby project a field projection on the surface that is continuously seen by the user during ambulation and is not dependent on stepping of the user during ambulation;
sensing weight bearing at the heel and forefoot of a shoe of the user to detect weight bearing at areas of the user's foot at heel contact and mid to late stance phase of a gait cycle during ambulation of the user; and
generating and transmitting an auditory cue to the user to notify the user that he or she has accomplished heel strike and forefoot weight transfer.

9. The gait facilitation and feedback method of claim 8, wherein the light beams are generated and projected with a unit supported on the surface.

10. The gait facilitation and feedback method of claim 8, wherein the light beams are generated and projected with a unit mounted on a belt worn by the user.

11. The gait facilitation and feedback method of claim 8, wherein the light beams are generated and projected with a unit attached to a shoe of the user.

12. The gait facilitation and feedback method of claim 8, wherein the auditory cue is transmitted to the user via a Bluetooth wireless earbud or speaker.

13. A gait facilitation and feedback system comprising:
means for generating and simultaneously projecting multiple light beams on a surface in front of a user's line of progression during ambulation by the user over the surface so that multiple illuminated lines are illuminated that are parallel to each other in front of and perpendicular to the user's line of progression during ambulation to thereby project a field projection on the surface that is continuously seen by the user during ambulation and is not dependent on stepping of the user during ambulation.

14. The gait facilitation and feedback system of claim 13, wherein the light beams are generated and projected with a unit adapted to be supported on the surface.

15. The gait facilitation and feedback system of claim 13, wherein the means for generating and projecting the field is a unit adapted to be mounted on a belt adapted to be worn by the user.

16. The gait facilitation and feedback system of claim 13, wherein the means for generating and projecting the field is a unit adapted to be attached to a shoe of the user.

17. The gait facilitation and feedback system of claim 13, further comprising means for sensing weight bearing at the heel and forefoot of a shoe of a user to detect weight bearing at areas of the user's foot at heel contact and mid to late stance phase of a gait cycle during ambulation of the user;

18. The gait facilitation and feedback system of claim 13, further comprising means for generating and transmitting an auditory cue to the user to notify the user that the user has accomplished heel strike and forefoot weight transfer.

19. The gait facilitation and feedback system of claim 18, wherein the means for generating and transmitting the auditory cue transmits the auditory cure to the user via a Bluetooth wireless earbud or speaker.
